(19) United States
(12) Patent Application Publication
(10) Pub. No.: US 2011/0033913 A1
(43) Pub. Date: Feb. 10, 2011

(54) METHOD AND APPARATUS FOR THEMICROBIOLOGICAL REMOVAL OF MERCURY FROM CONTAMINATED MATERIALS,

(76) Inventors: Giuseppina Bestetti, Agrate Brianza (IT); Isabella Gandolfi, Busto Arsizio (IT); Andrea Franzetti, Travedona Monate (IT)

Correspondence Address: Beusse Wolter Sanks Mora & Maire, 390 N. ORANGE AVENUE, SUITE 2500, ORLANDO, FL, 32801, US

(21) Appl. No.: 12/936,753

(22) PCT Filed: Apr. 7, 2009
(86) PCT No.: PCT/IB2009/051457

§ 371 Date: Oct. 25, 2010

(30) Foreign Application Priority Data
Apr. 7, 2008 (IT) RM2008A000183

(51) Int. Cl. A62D 3/02; C12N 1/20; C12M 1/00

(52) U.S. Cl. 435/252.5; 435/253.3; 435/262.5; 435/252.1; 435/289.1

(57) ABSTRACT

The present invention relates to microorganisms able to reduce mercury ion to metallic mercury; in particular, it refers to systems, apparatuses such as a stirred bioreactor and methods for microbiological mercury removal from contaminated materials, such as, e.g., contaminated environmental matrices, like soil and sediments. The contaminated material is mixed with selected microrganisms capable to enable enzymatic reduction of mercury in ionic form to elemental mercury.

### TECHNICAL FIELD

The present invention pertains to the field of mercury removal from materials. In particular, it refers to systems, apparatuses and methods for microbiological mercury removal from contaminated materials, such as, e.g., contaminated environmental matrices, like soil and sediments.

### STATE OF THE ART

Technologies based on microorganism use, allowing mercury removal and recovery, above all when the matrix to be treated be comprised of contaminated waters, are known. Some treatment systems provide mercury accumulation inside genetically engineered microbial cells, which are removed at the end of the treatment, thereby allowing mercury removal from the contaminated matrix (see references 1, 2).

Other technologies instead are based on microbiological mercury reduction by enzymatic way, yielding mercury in elementary form, more easily removable from the contaminated matrix with respect to its ionic forms. Some applications of these latter technologies to contaminated wastewater treatment are known in the literature, carried out in different scales, from small-batch systems (see reference 3) to fed-batch fermenters and larger-sized chemostats (see reference 4). Such a process has been developed to pilot scale: the system consists of a bioreactor with a volume of 0.7 m³, capable of treating wastewater outlet from a small industrial plant (see reference 5).

The above technologies are essentially based on the use of microorganisms, even genetically modified ones, inside a closed and controlled bioreactor, in which cells are mostly immobilized as biofilm on media consisting of various inert materials. Mercury reduced to its elementary form by microorganisms is accumulated inside the bioreactor or removed by air flow and collected into suitable traps, generally consisting of activated carbon. Microorganism growth in the biofilm is controlled by providing nutrients in suitable amounts.

Still fewer are the attempts made to apply microbial mercury reduction abilities to the reclamation of contaminated soils and sediments (see reference 6). In such a case, there are known only applications made of simple systems in flasks, into which soil to be treated and microorganisms are placed. Here as well, reduced mercury is collected in -traps placed at the system outlet.

According to what is known to the Inventors, the most advanced technique with concern to the treatment of mercury-contaminated soils (see references 7, 8) consists of an apparatus made of a Drechsel bottle containing contaminated sediments, treated beforehand with chemical compounds that solubilize mercury as much as possible, and an inoculation of microorganisms. The apparatus is crossed by an air flow that removes reduced mercury, which is then collected in a trap placed downstream of the system. However, the technology already developed provides a step of leaching the mercury with chemical compounds, preceding the step of biological metal reduction, whose drawbacks mainly consist in the high cost of the reactants used and the altering of matrix features. Moreover, the treatment already developed is almost exclusively focused on the removal of a single mercury compound, HgS, present in particular in anaerobic sediments, very scarcely soluble and chemically stable, therefore scarcely bioavailable, whereas it offers no solution for the removal of other forms of mercury, more abundant, e.g. in anaerobic environments, more mobile and therefore potentially more bioavailable.

Scope of the present invention is to remove the drawbacks of the prior art.

### SUMMARY OF THE INVENTION

The invention proposes a treatment comprised of a single step, in which microorganisms remove the fraction of mercury most bioavailable, and therefore potentially more toxic, in matrices coming from aerobic as well as anaerobic environments.

A first object of the present invention is a method for the removal of mercury in ionic form from a material. In particular, according to the present invention, said method comprises the step of mixing said material with at least one of the microorganisms described herein, for a time and under conditions suitable to allow enzymatic reduction of said mercury in ionic form to mercury in elementary form. In particular, the material is not subjected to any chemical modification pretreatment of the mercury present as contaminant. The method may further comprise the removal of said mercury in elementary form from said material.

A second object of the present invention is microorganism able to reduce mercury in ionic form to mercury in elementary form.

A third object of the present invention is the use of at least one species of the above-indicated microorganisms for mercury removal from a contaminated material.

A fourth object of the present invention is a system for biological mercury removal from a contaminated material. In particular, according to the present invention, such a system comprises: a bioreactor, apt to allow contact between said contaminated material and the above-indicated microorganisms for a time and under conditions such as to allow reduction of mercury in ionic form to mercury in elementary form; and such microorganisms.

A fifth object of the present invention is a method for preparing a culture of microorganisms belonging to the genus Bacillus, able to reduce mercury in ionic form to mercury in elementary form. In particular, according to the present description, such a method comprises the step of preparing a culture of said microorganism for a time and under conditions such as to obtain a cell density corresponding to a predetermined density, so as to maximize reduction of mercury in ionic form to mercury in elementary form by said microorganism.

With respect to methods known in the art, the microorganisms, uses, methods and systems of the present invention can be made so as to allow removal of a broad group of mercury compounds. In fact, mercury compounds removable with applications indicated in the present description comprise not only inorganic salts of mercury, like for instance HgCl₂, but also organic compounds of mercury, known to be more toxic and potentially more bioavailable, such as methylmercury. Moreover, the microorganisms, uses, methods and systems of the present description can be used so as to allow the treatment of contaminated material in a single stage, and therefore omit a pretreatment consisting in leaching the mercury with chemical compounds, which is generally associated to high costs due to reactants used and the possible altering of the matrix features.

Advantages offered by the present invention are those of allowing: a) prevalent removal of the more bioavailable mercury fraction, potentially more hazardous; b) option of treating a greater amount of material in the course of a single treatment; c) option of reusing the treated matrix, as its features are not altered by the treatment; d) economic saving, due to the elimination of the chemical leaching step, which envisages the use of costly reagents and the use of a lesser amount of water per soil gram.

The applications of the present invention will be better described with the aid of the annexed figures. Further peculiar embodiments, and advantages of the microorganisms, uses, methods and systems indicated herein will be made evident from. the description, drawings and claims.

### DESCRIPTION OF THE FIGURES

The annexed figures, which are incorporated in and constitute an integral part of this description, illustrate one or more embodiments of the present invention and, in conjunction with the detailed description, explain the principles and the embodiments of the present invention.

FIG. 1 shows a schematic depiction of a system for mercury removal from a matrix .according to some embodiments of the present description.

FIG. 2 shows a schematic depiction of a bioreactor according to some embodiments of the present description.

FIG. 3 shows a schematic depiction of a bioreactor according to some embodiments of the present description.

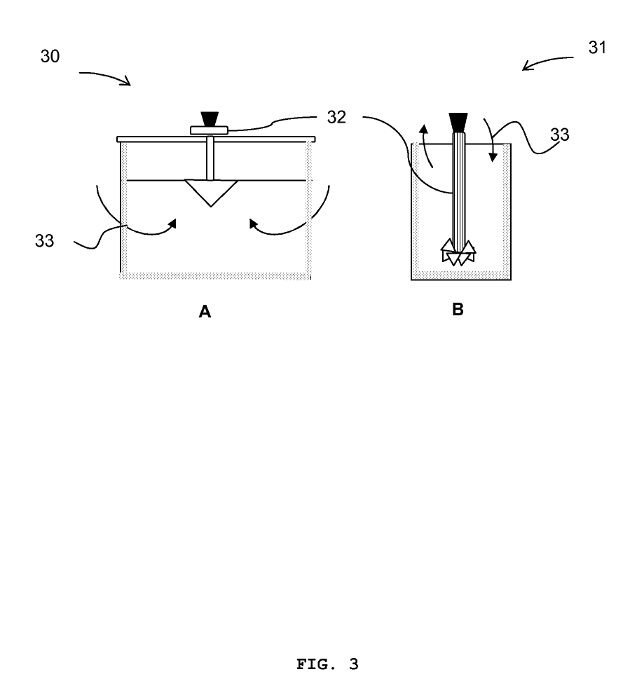

Alike symbols in the various drawings denote alike elements.

### DETAILED DESCRIPTION

Microorganisms

The microorganisms according to the present invention belong to various genera of bacteria able to produce the enzymes needed to allow access of mercury compounds into the cell and their reduction. Therefore, they are able to enzymatically reduce mercury in ionic form to mercury in elementary form. Such microorganisms are selected among the genera: Aeromonas, Acinetobacter, Alcaligenes, Bacillus, Flavobacterium, Pseudomonas, Rhodococcus.

In a specific embodiment, of the invention the microorganisms belong to the genus Bacillus, in particular the strain deposited, in accordance with the Budapest Treaty, on Mar. 25, 2008, at the BCCM/LMG Bacteria Collection—Laboratorium voor Microbiologie—Universiteit Gent—Gent (Belgium), with the accession number LMG P-24567.

Microorganism preparation is carried out by cultivating an adequate amount of microorganisms belonging to the genus Bacillus, until obtaining the initial cell density desired in the aqueous phase. The culture medium preferably consists of complete media, containing protein extracts.

Material to be Decontaminated

The systems, methods and uses described herein are based in particular on the natural abilities of said microorganisms to enzymatically reduce the mercury in ionic form, preferably mercury II (Hg²⁺) to the elementary form.

In the absence of further qualification, the term “mercury” to the ends of the present description is to be understood as comprising both mercury in elementary form (identified in the present description also as mercury 0 or metallic mercury) and mercury in ionic form (herein also identified as mercury +1 or +2), the latter comprising ions Hg₂²⁺ and Hg²⁺ as well as the related salts or organic compounds including such ions, like, e.g., ionizable salts of mercury (e.g., HgCl₂), usually soluble, and organomercurial compounds, such as alkyl- or aryl- derivatives of mercury (e.g., CH₃Hg).

The term “bioavailable” related to the mercury compound denotes compounds that can easily enter and/or accumulate in living organisms, owing to their high solubility or affinity with hydrophobic compounds of the organisms.

The term “material” as used in the present description denotes any one undifferentiated substance that may be subject to mercury contamination.

The term “matrix” to the ends of the present description is to be understood as extending to any one system comprising the contaminated material, solid-, semisolid- or liquid-phase matrices included, and includes, by way of a non-limiting example, matrices such as soils, rocks, sediments, filtering materials and/or absorbent materials.

The term “contaminated” as used in the present description with reference to a material, and to a matrix, denotes the presence, in said material, of mercury as defined in the present description at concentrations higher than those envisaged as limit by the laws in force, quantifiable with methods, technologies and/or instruments identifiable by a person skilled in the art.

Methods

The method according to the invention comprises a step in which there are mixed at least one of the above-mentioned microorganisms with a material or a matrix containing mercury, and in particular mercury in ionic form, for a time and under conditions suitable to allow enzymatic reduction of mercury in ionic form to mercury in elementary form by the microorganisms. Mercury removal from the matrix, by means of the microorganisms identified in the present description, is carried out by a method in which treatment parameters can be optimized to maximize mercury removal.

In some embodiments, contact is effected by resuspending the microorganisms and the matrix in a single aqueous solution containing the chemical elements necessary to microbial metabolism and for a time such as to optimize also the growth of said microorganisms on said matrix. The method further comprises the step of removing the mercury in elementary form from the matrix treated with the microorganisms.

The method described herein is essentially a one-step method. By the wording “one-step” it is meant a method comprising no step of pretreating the contaminated material or matrices, aimed to the chemical modification and/or bioavailability of the mercury present as a contaminant. Therefore, the method envisages no preliminary treatments of the material or of the matrices, such as acid leaching or transformation of mercury-containing species, e.g. oxidations, into more soluble compounds.

The step of mixing the microorganisms with the above-mentioned matrix is carried out by preparation of a culture of said microorganism for a time and under conditions such as to attain a cell density corresponding to a predetermined density, followed by subsequent contact of said culture with the material to be decontaminated. In particular, cell density is predetermined so as to maximize reduction of mercury in ionic form to mercury in elementary form by the microorganism when brought into contact with the material to be decontaminated. Preferably, optimal cell density is attained by cultivating the microorganisms on complete media containing protein extracts, for a time needed to attain optical density values of the culture no lower than 1 AU (Absorbance Unit), measured at 600 nm.

In some embodiments the solid matrix is suspended in a liquid phase, resulting in a semisolid phase called slurry.

The material or the solid matrix is mixed to an amount of liquid phase, e.g. water, no lower than three times the weight of the matrix to be treated, so as to obtain a semisolid phase, which can be more easily homogenized with respect to the solid phase. The amount of liquid phase can be of from 3 to 20 times, preferably 5, 8, 10, 15 times the weight of the solid.

Contact with microorganisms is made possibly in the presence of further substances and/or compounds apt to allow or facilitate their growth and/or the enzymatic reduction of mercury in ionic form.

Such substances or compounds can be dissolved in the liquid phase of the suspension and comprise mixtures of mineral salts in amounts sufficient to maintain the medium salinity that is most effective for microorganism activity. In particular, the suspension can be additioned with mixtures, mainly of nitrates and phosphates, which may be prepared for the purpose or consist of already marketed sources of nitrogen and phosphor, like e.g. fertilizers utilized in agriculture. Preferably, nitrogen and phosphor concentrations respectively range between 10, and 50 mM and between 10 and 100 mM.

Moreover, there may be added thiolic compounds such as sodium thioglycolate, cysteine, glutathione or mercaptans, in concentrations ranging from 1 to 20 mM, for instance 10 mM, which be able to increase synthesis and activity of enzymes catalyzing the mercury reduction process, so as to put the microorganisms in conditions under which the highest viable efficiency may be obtained. Finally, there may be added also various types of compounds, e.g. surfactants, having the property of facilitating desorption and solubilization of mercury adsorbed on solid particles of the matrix, without changing its chemical nature, in order to foster the microbial reduction process, making mercury itself more available for the microorganisms. Such substances can be added in a concentration of from 1 to 10 g/l, e.g. 5 g/l.

Moreover, there may be added simple carbon sources, such as glucose, sucrose, etc., in a concentration ranging from 1 to 10 g/l to foster microbial growth.

Such substances and compounds can be brought into contact with the matrix to be decontaminated and/or the microorganisms before, or after contact between microorganisms and matrix. In some embodiments, the matrix is mixed with said further substances and compounds before contact with the microorganisms. Matrix pretreatment can be carried out directly inside the bioreactor, before microorganism addition, or by homogeneizing with mechanical means the matrix and the compounds to be added prior to introduction in the bioreactor.

Contact between matrix and microorganisms, and optionally also with the above-indicated substances and compounds, may be optimized, for instance by stirring means apt to allow or facilitate diffusion of the microorganisms and, possibly, of the further materials and compounds, on the matrix to be decontaminated.

Removal of mercury in elementary form, a volatile chemical species, may be carried out by a gas flow through the reaction mixture. E.g., there may be used a flow of an oxygen-containing gas mixture, like a flow of air, preferably humidified to maintain the humidity features of the treated slurry. The gas flow removes and transfers mercury from the matrix to a trap containing a support (i.e. a material apt to immobilize mercury) in which removed mercury be accumulated to be subsequently disposed of or recovered.

Any mercury fraction remaining in solution in the aqueous phase at the end of the treatment, and that has not been removed by the microorganisms, can be separately disposed of, after concentration in a small volume, so as to obtain the maximum possible yield of removal of the mercury compounds from the treated matrix.

Trap-collected mercury is periodically quantitated by atomic absorption spectroscopy. Final residual concentration of mercury in both phases, liquid and solid, of the slurry is measured at the end of the treatment, so as to calculate a mass balance, to check that the entire fraction of microorganism-reduced mercury be collected in the traps. Moreover, the fraction of bioavailable mercury is quantitated, with a suitable methodology, before and after the treatment, in order to assess reduction of the hazardousness of the treated matrix.

Bioreactor

Matrix decontamination from mercury can be carried out in a bioreactor apt to contain the microorganisms indicated herein, together with the matrix itself. The bioreactor is part of a system using the microorganisms described herein for, possibly, mercury recovery from the matrix itself.

In accordance with the present invention, such a system contains a) a bioreactor inside which the treatment Occurs; together with b) a system for stirring the material contained in the bioreactor; c) a system for transit of fluid used for mercury removal from the bioreactor and/or d) a support for immobilization of mercury removed from the bioreactor through forced ventilation.

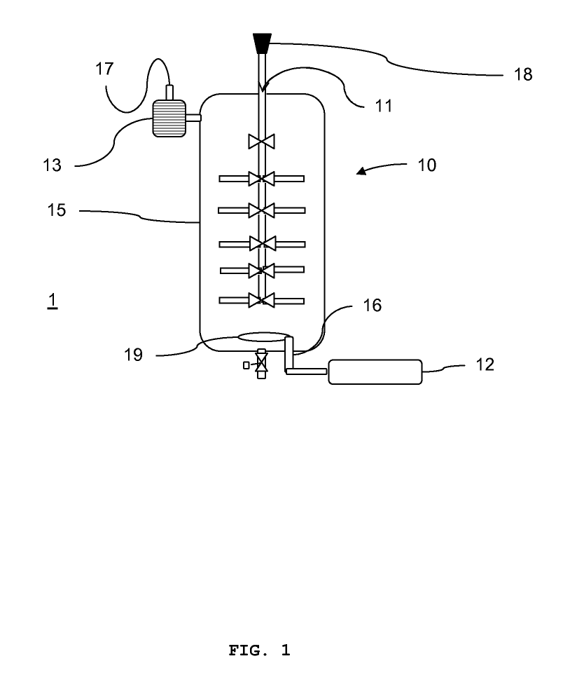

A specific embodiment of the system is illustrated in FIG. 1, wherein it is depicted a system (1) comprising
a closed bioreactor (10), inside which the treatment occurs;.
a system (11) for stirring the material contained in the bioreactor;
a forced ventilation system (12) allowing . oxygen contribution and microorganism-reduced mercury removal;
a trap (13) downstream of the bioreactor, for trapping mercury removed by the system;

the bioreactor (10) may be a continuous stirred bioreactor with a blade rotor, allowing continuous mass/fluid (gas/liquid) redistribution, together with heat transfer inside the bioreactor in which the content is mixed.

Such a bioreactor may be comprised of a container (15) with fluid-tight walls, made of a material that does not adsorb mercury, which can be hermetically sealed after introduction of the contaminated material to be treated, with the exception of air flow inlet (16) and outlet (17). The air flow that is being outlet transits through the trap (13). The treatment therefore occurs preferably in a batch. The bioreactor may be of variable volume and piece-formed, or made of a main body and a lid fastenable so as to obtain a tight seal in order to prevent mercury dispersal by volatilization.

In some embodiments of the system illustrated in the present description the bioreactor contains an optionally adjustable stirring system, allowing to keep as homogeneous as possible the mixture comprised of the solid material to be treated and water, optionally additioned with compounds fostering the biological process (such a mixture being identified in the present description also by the term “slurry”). In the bioreactor (10) illustrated in FIG. 1, a blade stirring system (11) is used whose rotation is maintained by a motor (18).

Upstream of the bioreactor a system (12) is placed which guarantees forced ventilation of the system and a flow not lower than a preselected value. Inlet air should always be humidified in order to guarantee constant water content inside the bioreactor. In particular, air flow can be maintained by systems such as pumps or compressors, maintaining a known and possibly constant flow rate. Said flow can be introduced in the system by means of diffusers of various type, generally immersed in the slurry, such. as, e.g., the diffuser (19) allowing a more effective oxygenation.

Downstream of the bioreactor a trap (13) is placed, comprised of a support containing strong oxidizers or of activated carbon, allowing to accumulate and recover mercury removed from the treated matrix.

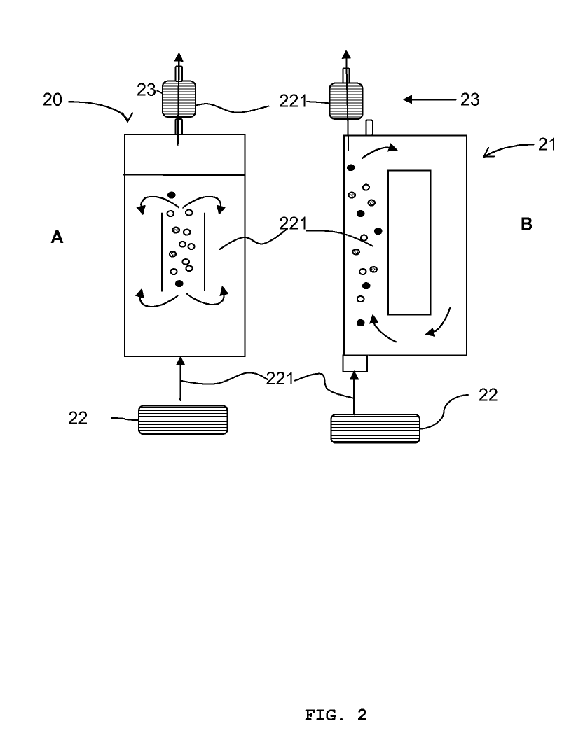

The bioreactor may be comprised of a closed-cycle bioreactor, like e.g. bioreactors (20) and (21) schematically illustrated respectively in FIGS. 2A and 2B, in which the stirring system is comprised of a system (22) for pumping air inside the reactor, allowing generation of a possibly adjustable and constant air flow (221). The bioreactors (20) and (21) exploit air diffusion to generate a forced and controlled flow of liquid in the bioreactor, with the further advantage of allowing lower energy consumption. The pumping system (22) of the bioreactors (20) and (21) can, e.g., be comprised of a mechanical system or a pneumatic system (e.g., compressed-air pumping system of the bioreactor). In this bioreactor as well, the outlet flow transits through a trap (23).

The microorganisms, methods, uses and systems, bioreactors and apparatuses described herein find application for: a) removal of organic and inorganic compounds of mercury in contaminated soils and sediments; b) reclamation with a treatment ex situ of contaminated sites, in which the main contamination be from mercury, c) mercury concentration in small volumes of material, so as to facilitate its disposal, or recovery of metallic mercury, which can thus be reused; and/or d) reuse and recovery of treated matrices, once decontaminated.

The microorganisms, uses, systems, methods described herein will be illustrated hereinafter, in some of their aspects, by means of specific examples relating to the experimental steps of preparing and assessing mercury removal from matrices to be decontaminated. These examples are merely for illustration, and in no way limit the scope of the claims.

### EXAMPLES

Some aspects of the present description will be further illustrated with the aid of the following examples:

### Example 1

Mercury removal from a soil contaminated with HgCl₂ at a concentration of 100 mg/kg was conducted in slurry phase in a 1-liter volume bioreactor, equipped with a blade stirrer connected to a motor for maintaining slurry homogeneity; stirring was kept constant at 150 rpm in all tests. Air flow, maintained by a pump external to the bioreactor, is inlet by means of a porous septum of dimensions slightly smaller than the bioreactor diameter, positioned on the bottom of the bioreactor itself; Inlet air flow rate was kept constant at 1 L/min.

Downstream of the bioreactor there were positioned two traps in series, each consisting of 50 mL of 5% H₂SO₄ and 0.6% KMnO₄ solution, in which mercury stripped by the air flow was collected. Traps were periodically replaced and analyzed to determine mercury concentration.

The test ended at +144 h and percentage of residual mercury in both phases, solid and liquid, was determined. Moreover, percentage of bioavailable mercury was determined, with respect to the total, the initial time and the final time, by using the following methodology: 5 g soil were placed in a beaker with 10 mL extracting solution (DTPA 1.97 g/L, CaCl₂.2 H₂O 1.46 g/L, triethanolamine 14.92 g/L) and left under stirring for 2 hours. Slurry was then centrifuged at 5000 rpm for 5 min; supernatant was filtered on filter paper and analyzed. All mercury analyses were performed by using a mercury analyzer based on atomic absorption spectrometry.

Microorganism inoculation consisted of a culture of Bacillus sp. RM1, cultivated overnight in rich medium (tryptone 10%, yeast extract 5%, NaCl 5%) and resuspended in the aqueous phase of the slurry so as to obtain a cell optical density, measured at 600 nm, equal to 1.

Soil/water ratio was set at 1:10; to the aqueous phase there was added a mixture of mineral medium thus composed: Na₂HPO₄ 7 g/L, KH₂PO₄ 3 g/L, NaCl 0.5 g/L, NH₄Cl 1 g/L. Moreover, sodium thioglycolate was added, at a concentration of 10 mM, referred to the aqueous phase. This test yielded a soil mercury removal percentage equal to 67±7%, whereas the residue in the solid phase at the end of the treatment was equal to 20±6%. The fraction of bioavailable mercury present in the soil, equal to 18.9±0.4% before the treatment, was reduced to 3.4±0.6% at the end of the treatment.

### Example 2

Mercury removal from a soil contaminated with HgCl₂ at a concentration of 40 mg/kg was conducted as described in the preceding example. In addition, liquid phase was additioned with a solution of a compound exhibiting biosurfactant action, a rhamnolipid available on the market, at a concentration of 5 g/L. This test yielded a soil mercury removal percentage equal to 47±9%, whereas residue in solid phase at the end of the treatment was equal to 40±9%. The fraction of bioavailable mercury present in the soil, equal to 14.3±1.5% before the treatment, was reduced to 8.6±1.0% at the end of the treatment.

It is understood that the present description is not to be limited to specific configurations of the apparatus, to specific materials, applications or systems, which of course may vary.

Moreover, it is understood that the terminology used in the present application, which has been used in order to describe specific embodiments, is not to be understood as limitative.

Unless otherwise defined, all technical and scientific terms used in the present description have the same meaning usually understood by a person skilled in the art to which the description pertains. Though any method or material alike or equivalent to the described ones may be used to carry out the invention, specific materials and methods are described by way of example.

The full description of each document cited is by all means to be understood as repeated and transcribed in its entirety in the present application.

### Example 3

Mercury removal from a soil contaminated with HgCl₂ at a concentration of 100 mg/kg was conducted in slurry phase in a 1 liter-volume bioreactor, as described in example 1.

Microorganism inoculation consisted of a culture of Pseudomonas fluorescens, cultivated overnight on rich medium (tryptone 10%, yeast extract 5%, NaCl 5%) and resuspended in the aqueous phase of the slurry so as to obtain a cell optical density, measured at 600 nm, equal to 1.

Soil/water ratio was set at 1:10; to the aqueous phase there was added a mixture of mineral medium thus composed: Na₂HPO₄ 7 g/L, KH₂PO₄ 3 g/L, NaCl 0.5 g/L, NH₄Cl 1 g/L. Moreover, sodium thioglycolate was added at a concentration of 5 mM, referred to the aqueous phase.

This test yielded a soil mercury removal percentage equal to 53±18%, whereas the residue in the solid phase at the end of the treatment was equal to 37±17%. The fraction of bioavailable mercury present in the soil, equal to 30.9±9.7% prior to the treatment, was reduced to 2.8±1.2% at the end of the treatment.

### Example 4

Mercury removal from a soil contaminated with HgCl₂ at a concentration of 40 mg/kg was conducted as described in Example 1, with the difference that as inoculation a culture of Pseudomonas fluorescens was used, rather than a culture of Bacillus sp. RM1. The Pseudomonas fluorescens culture was prepared as described in Example 3. Moreover, the liquid phase was additioned with a solution of a compound exhibiting biosurfactant action, a rhamnolipid present on the market, at a concentration of 5 g/L. This test yielded a soil mercury removal percentage equal to 51±8%, whereas the residue in the solid phase at the end of the treatment was equal to 23±5%. The fraction of bioavailable mercury present in the soil, equal to 23.9±5.9% before the treatment, was reduced to 14.4±1.0% at the end of the treatment.

### REFERENCES

[1] Chakrabarty A M, Friello D A, Mylroie J R. 1975. Mercury concentration by the use of microorganisms. U.S. Pat. No. 3,923,597.

[2] Kiyono, M, Pan-Hou H. 2006. Genetic engineering of bacteria for environmental remediation of mercury. J Health Sci 52:199-204.

[3] Chang J-S, Law W-S. 1998. Development of microbial mercury detoxification processes using mercury-hyperresistant strain of Pseudomonas aeruginosa PU21. Biotechnol Bioeng 57:462-470.

[4] Okino S, Kazuhiro I, Osami Y, Tanaka H. 2000. Development of a biological mercury removal-recovery system. Biotechnol Lett 22:783-788.

[5] Wagner-Döbler I, von Canstein H, Li Y, Timmis K N, Deckwer W-D. 2000. Removal of mercury from chemical wastewater by microorganisms in technical scale. Environ Sci Technol 34:4628-4634.

[6] Hansen C L, Stevens D K, Warner D N, Zhang S. 1992. Biologically enhanced removal of mercury from contaminated soil. Proceedings of “85^{th} Annual Meeting and Exhibition of Air and Waste Management Association”.

[7] Nakamura K. 1998. Treatment of mercury-polluted material, and microorganism especially useful. for the treatment. Patent JP10229873.

[8] Nakamura K, Hagimine M, Sakai M, Furukawa K. 1999. Removal of mercury from mercury-contaminated sediments using a combined method of chemical leaching and volatilization of mercury by bacteria. Biodegradation 10:443-447.

1-25. (canceled)

26. A method for the removal of mercury in ionic for from a solid or semisolid material, said methods comprising steps wherein:
said material is mixed with at least one microorganism selected from the genera Aeromonas, Acinetobacter, Alcaligenes, Bacillus, Flavobacterium, Pseudomonas, Rhodococcus, able to reduce mercury in ionic form to mercury in elementary form, for a time and under conditions suitable to allow enzymatic reduction of said mercury in ionic form to mercury in elementary form, and said mercury in elementary form is removed from said material and wherein the material is not subjected to any chemical modification pretreatment of the mercury present as contaminant.

27. The method according to any one of the claim 26, wherein the microorganism is a strain of Bacillus deposited at the BCCM/LMG Bacteria Collection—Laboratorium voor Microbiologie—Universiteit Gent—Gent (Belgium) on Mar. 25, 2008 with the accession number LMG P-24567.

28. The method according to claim 26, wherein the material consists of a matrix in a solid, semisolid form.

29. The method according to claim 28, wherein the material is a suspension of soil, sediments or other matrix in an aqueous phase.

30. The method according to claim 29 wherein the aqueous phase is additioned with mixtures of mineral salts, thiolic compounds, and optionally surfactants.

31. The method according to claim 30, wherein the aqueous phase is present in an amount not lower than three times the weight of the solid material to be treated.

32. The method according to claim 26, comprising the step in which it is prepared a culture of said microorganism for a time and under conditions such as to attain an optical density of the culture no lower than 1 AU (Absorbance Unit), measured at 600 nm, corresponding to a density maximizing the reduction of mercury in ionic form to mercury in elementary form by said microorganism.

33. The method according to claim 26, wherein said mercury in elementary form is removed through a forced ventilation system apt to bring a gas flow into contact with the material.

34. The method of claim 33, wherein the gas flow is humidified air.

35. The method according to claim 26, wherein subsequently to the removal from said material said mercury in elementary form is recovered.

36. The method according to claim 35, wherein the recovered mercury is quantitated by measurement of the final residual concentration of mercury, both in solid phase and in liquid phase, so as to calculate a mass balance, by means of atomic absorption spectroscopy.

37. A microorganism able to reduce mercury in ionic form to mercury in elementary form, said microorganism belonging to the genus Bacillus.

38. The microorganism according to claim 37, said microorganism being deposited at the BCCM/LMG Bacteria Collection—Laboratorium voor Microbiologie—Universiteit Gent—Gent (Belgium) with the accession number LMG P-24567.

39. Use of microorganisms according to claim 38, for mercury removal from a material.

40. The use according to claim 39, wherein said material consists of a matrix in solid, semisolid or liquid form.

41. An apparatus for biological mercury removal from a contaminated material, comprising:
a bioreactor, apt to allow contact between said contaminated material and at least one of the microorganisms according to claim 13 for a time and under conditions such as to allow reduction of mercury in ionic form to mercury in elementary form,
a forced ventilation system, apt to contribute a fluid for removal of mercury in elementary form, and
a trap downstream of the bioreactor for trapping the mercury in elementary form once removed from the contaminated material.

42. The apparatus according to claim 41, wherein the ventilation system comprises a fluid inlet made on the bioreactor, a fluid outlet made on the bioreactor, and a fluid flow apt to run between said inlet and said outlet.

43. The apparatus according to claim 42 further comprising a system for stirring the material contained in said bioreactor.

44. The apparatus according to claim 41, wherein the means for stirring the material comprises a rotary blade system.

45. The apparatus according to claim 41, wherein the trap comprises strong oxidizers or activated carbons.

46. The apparatus according to claim 41, wherein the bioreactor is a variable volume bioreactor.

47. A method for preparing a culture of microorganisms belonging to the genus Aeromonas, Acinetobacter, Alcaligenes, Bacillus, Flavobacterium, Pseudomonas or Rhodococcus, and able to reduce mercury in ionic form to mercury in elementary form, said method comprising the steps wherein:
it is prepared a culture of said microorganism in a medium consisting of complete media, containing protein extracts, for a time and under conditions such as to attain an optical density of the culture no lower than 1 AU (Absorbance Unit), measured at 600 nm, corresponding to a density maximizing the reduction of mercury in ionic form to mercury in elementary form by said microorganism.
